(19) United States
(12) Patent Application Publication
(10) Pub. No.: US 2014/0024736 A1
(43) Pub. Date: Jan. 23, 2014

(54) POLYMER ARTICLES HAVING CHEMICALLY BONDED AGENTS AND METHODS OF MAKING THE SAME

(71) Applicants: Brian H. Thomas, Auburndale, FL (US); Dirk Pletcher, Walkerton, IN (US); Donald L. Yakimicki, Warsaw, IN (US)

(72) Inventors: Brian H. Thomas, Auburndale, FL (US); Dirk Pletcher, Walkerton, IN (US); Donald L. Yakimicki, Warsaw, IN (US)

(73) Assignee: Zimmer, Inc., Warsaw, IN (US)

(21) Appl. No.: 13/883,041

(22) PCT Filed: Nov. 2, 2011
(86) PCT No.: PCT/US11/58960

§ 371 Date: Sep. 20, 2013

Related U.S. Application Data
(60) Provisional application No. 61/409,750, filed on Nov. 3, 2010.
(60) Provisional application No. 61/426,873, filed on Dec. 23, 2010.

(51) Int. Cl. A61L 31/04

(52) U.S. Cl. CPC A61L 31/048
USPC 522/116; 525/333.7; 525/301; 525/293; 522/113; 522/120

(57) ABSTRACT

Modified polymeric articles having modifying agents dispersed within and bonded to an interior region of the article. Methods of modifying polymer materials used to form polymeric articles, and methods of making polymeric articles from polymer particles having modifying agents bonded thereto.

### CROSS-REFERENCE TO RELATED APPLICATIONS

This application claims the benefit of U.S. Provisional Patent Application Ser. No. 61/426,873 filed on Dec. 23, 2010 and U.S. Provisional Application Ser. No. 61/409,750 filed on Nov. 3, 2010; the disclosures of both of these applications are hereby incorporated by reference herein in their entireties.

### FIELD OF THE DISCLOSURE

The present disclosure relates to modified polymeric materials and methods of modifying polymeric materials. The modified polymeric materials may be further processed to produce polymeric articles that have modifying agents bonded to the surface of the article and/or bonded to an interior region of the article. The present disclosure also relates to modified polymer powders/particulates that may be employed to produce such articles.

### BACKGROUND

Implantable medical devices made in whole or in part from polymeric materials have been developed for implantation or insertion into the body. Examples of such medical devices include endoprosthetic joints, which typically include a metal or ceramic component articulating on or bearing against the polymeric surface of another article. One example of such an endoprosthetic device is a knee prosthesis that includes a femoral knee prosthesis which articulates against the polymeric surface of the corresponding part of the article or implant. The polymeric articles are typically made from, for example, polyethylene, ultra high molecular weight polyethylene (UHMWPE), polyaryletherketones or combinations and blends of such polymers.

In order to enhance certain characteristics of the polymeric article, (e.g., lubricity, hydrophobicity, hydrophilicity, crosslinking or wettability) the exterior surfaces of the polymeric article may be subjected to surface treatments. Such surface treatments are commonly applied to an already formed polymeric article, which has been formed by, for example, compression molding, ram extrusion or deposition. After the polymeric article has been formed, the exterior surface of the article is subjected to a surface modification process to modify the polymer molecules on or near the exterior surface of the polymeric article. Exterior surface modification of a polymeric article may be accomplished by, for example, plasma treatment or wet or dry chemical treatments of the polymeric article's exterior surface.

While such methods of imparting desirable properties to selected portions or surfaces of polymeric articles or the polymeric components of the medical implants have worked satisfactorily, introducing selected modifying agents into the starting polymeric materials used to make the polymeric article or polymeric component of an implant may provide other advantageous properties.

### SUMMARY

In one aspect of the present disclosure relates to an implantable medical device including a polymeric article having an outer surface and an interior region. The polymer molecules of the article include at least one selected modifying agent bonded thereto, wherein the polymer molecules are distributed within at least a portion of the interior region.

In another aspect, a polymer material for manufacturing a medical implant comprises a polymer powder including particles and a modifying agent bonded to the particles.

In yet a further aspect, a method of manufacturing a polymeric article includes bonding one or more modifying agents to molecules of polymer particles. The particles are then consolidated to form a polymeric article.

By the use of certain modifying agents, the polymer molecules can be made to crosslink without the use of radiation. Avoiding radiation can reduce the formation of persistent free radicals and vinyl groups in the polymer. Free radicals existing in the crosslinked polymer can reduce the life of the polymer.

The polymer article can experience wear in use and may eventually be worn away. By having polymer molecules with agents bonded thereto within middle portions, sections or layers of the polymeric article, as the exterior surface of the polymeric article is worn away, an inner region of polymeric article, which includes the modifying agents bonded thereto, becomes the new exterior surface. Thus, the polymeric article may be considered to have a renewable exterior surface. Additionally, different layers or portions of the polymeric article can have different agents bonded thereto such that as the polymeric article undergoes wear, new layers or sections having different agents bonded thereto will be exposed over time.

### BRIEF DESCRIPTION OF THE FIGURES

In the course of this description, reference will be made to the accompanying drawing(s), wherein:

FIG. 1 is an exploded perspective view showing the components of a knee replacement system including one example of a polymeric article;

FIG. 2 shows one embodiment of a crosslinking reaction between two polyethylene polymers, each having a crosslinking functional group;

FIG. 3 shows another embodiment of a crosslinking reaction between two polyethylene polymers, each polyethylene polymer having a crosslinking functional group and includes a bridging group;

FIG. 3A shows one embodiment of a crosslinking reaction between two polyethylene polymers, each polyethylene polymer having a crosslinking functional group and includes a bridging group and a mating group;

FIG. 4 shows one embodiment of a reaction adding a crosslinking group to a polyethylene polypropylene copolymer to reduce the number of tertiary hydrogens;

FIG. 5 shows one embodiment of a crosslinking reaction between two polyethylene polypropylene copolymers, each having a crosslinking functional group;

FIG. 6 shows one embodiment of a crosslinking reaction between two polyethylene polypropylene copolymers, each having a sulfonic acid crosslinking group;

FIG. 7 shows one embodiment of a crosslinking reaction between two polyethylene polypropylene copolymers, each having a trifluorovinyl crosslinking group;

FIG. 8 is a SEM image of a sample of a polymeric article comprised of UHMWPE and having silver dispersed throughout the article and bonded thereto;

FIG. 9 is another SEM image of the sample of FIG. 2 that has been configured to show the silver atoms dispersed within the sample; and

FIGS. 10 and 11 are schematic drawings of pucks made from consolidated polymer resin.

### DETAILED DESCRIPTION

As required, detailed embodiments of the present invention are disclosed herein; however, it will be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention in virtually any appropriate manner.

The polymeric materials and articles disclosed herein are particularly useful in the manufacture of medical implants or medical implant systems that are permanently or temporarily implanted within a human or animal body. The polymer molecules of the polymeric articles include one or more modifying agents that are bonded to the polymer molecules, thereby imparting the finished article with the desirable properties. The modifying agents may be antioxidants, or biological agents, functional or reactive agents or other agents for modifying the properties of the material and consequently, the finished article.

In one embodiment, the polymer molecules have a selected antioxidant bonded to the molecules. In another embodiment, the polymer molecules have a selected biological agent bonded thereto. In yet another embodiment, the polymer molecules have one or more different types of agents bonded thereto. For example, two or more different types of antioxidants may be bonded to the same polymer molecules, or two or more different types of biological agents may be bonded to the same polymer molecules, or one or more antioxidants and one or more biological agents may be bonded to the same polymer molecules.

In another embodiment the polymer molecules have one or more functional or reactive agents bonded thereto. The functional or reactive agents may serve as binding locations or to provide bridging or spacing for other agents. For example, the functional or reactive agent can act as a bridging group by forming a chemical bond with an antioxidant, biologic agent, or other functional or reactive agent which may not readily bond with the polymer itself. The functional or reactive agents may also serve other functions, such as to increase lubricity or provide crosslinking. For example the functional or reactive agent can be a crosslinking group that can be bonded to the polymer molecules and then made to react and form bonds with an adjacent crosslinking group. In this manner crosslinks can be formed without the use of radiation treatments. Preferably the functional or reactive agent is present in a majority of the polymer monomers that make up the polymeric article. Alternatively, the functional or reactive agent can be present in a minority of the polymer monomers that make up the polymeric article, for example from between 0.1 to 10 mole percent of the polymer monomers.

The bond between the agent and the polymer molecules may be such that the agent is substantially non-leachable from the article, i.e., the agent is not readily drawn out of the polymer article. However, the article also may include, if desired, leachable agents that are bonded to the article and/or leachable agents that are not bonded to the article.

The antioxidants may be any antioxidant, such as tocopherol (Vitamin E), unsaturated tocopherols, such as tocotrienols, Vitamin C, lycopene, honey, phenolic antioxidants, amine antioxidants, hydroquinone, beta-carotene, ascorbic acid, CoQ-enzyme, and derivatives thereof. Additionally, as any tocopherol may be used, such as d-α-tocopherol, d,l-α-tocopherol, or α-tocopherol acetate, unless otherwise specifically stated herein, the term “tocopherol” and “Vitamin E” in its generic form refers to all tocopherols.

The biological agents may include, but are not limited to, antimicrobials, antibiotics, anti-inflammatories, steroids or other suitable agents that have biological effects. The antimicrobials may include silver, cooper or zinc ions.

As discussed above, reactive or functional agents may include bridging groups and crosslinking groups. Bridging and binding groups may include but are not limited to acrylates, amines, amides, imines, imides, hydroxyls, carbonyls, aldehydes, carboxylates, carboxyls, ethers, esters, sulfonics, epoxides, alkanes, alkyl ethers, alkyl esters, perfluoroalkyl, and aromatic groups, also oligomers or polymers such as low molecular weight polyethylene glycol, polyethylene, polymethacrylic acid, polyacrylamide.

Crosslinking groups can be selected from but not limited to acrylates, amines, carboxylics such as carboxylic acids, alcohols, trifluorovinyls, amides, sulfonics such as sulfonic acids, phosphorics such as phosphoric acids, cyano, and epoxide groups.

Crosslinking groups can be selected based on preferred reaction schemes. Accordingly, many different crosslinking functional groups may be used. Preferably, the reaction of the crosslinking groups to bond with each other and form crosslinks does not require complicated reaction steps and more preferably does not require additional expensive, exotic or biologically harmful reactants. In one embodiment, the crosslinking groups can be selected such that the crosslinking reaction would not require any additional reactants. In another embodiment, the crosslinking groups can be selected such that the crosslinking reaction can be accomplished by heating. In yet another embodiment, the crosslinking groups can be selected such that the crosslinking reaction can be carried out by heating to a temperature at or below the melting point of the polymer.

In one embodiment, the heating can be performed in a heating step. Alternatively, the heating may be provided as a result of any consolidation steps performed on the polymer such as compression molding, sintering, injection molding or extrusion. In another embodiment the heating can be applied in addition to any consolidation steps.

Preferred crosslinking groups can react and form bonds with each other in (or under the influence of) a thermal reaction process without additional reactants. Preferably, the heat required to initiate the reaction and form crosslinking bonds can be at or below the melting point of the polymer. In one embodiment the heat required to react the crosslinking groups to form crosslink bonds can be at or below 180° C., preferably at or below 150° C., and more preferably at or below 120° C.

The crosslinking groups of the polymer molecules can react with each other without the use of additional reactants or complex reaction steps and without having to subject the polymer to radiation. Preferably, the crosslinking groups can react by thermal activation such as by heating. The reaction between the crosslinking groups may involve cyclodimerization, dehydration, condensation, or addition reactions. In one embodiment, the crosslinking groups are selected from but not limited to sulfonic acid groups, trifluorovinyl groups, phosphoric acid groups, carboxylic acid groups, epoxides, and cyano groups.

The polymer molecules including agents bonded thereto may be present at the outer or exterior surface of the article and/or in the interior regions of the polymeric article. In one embodiment, such polymer molecules are distributed throughout the polymeric article so that the agent is dispersed throughout the article. As used herein, “throughout” refers to distribution of the agents across substantially the entire article including uniform or substantially uniform distribution and varied or irregular distribution of the agents in the polymeric article.

Alternatively, the polymer molecules having agents bonded thereto may be selectively located, distributed or dispersed in particular sections, portions, surfaces or layers so that the polymeric article only includes agents within selected portions, sections, surfaces or layers of the polymeric article. For example, the article may include a first section or layer that includes polymer molecules having agents bonded thereto, and a second section or layer that includes molecules that do not have any agents bonded thereto.

Further, different polymer molecules having different agents bonded thereto may be combined together in the same portion or layer of the polymeric article. Alternatively, each portion or layer of the article may include polymer molecules having a particular agent or combination of agents, which is different from the agent or combination of agents of the adjacent portion or layers. The polymeric article may be layered so that as the exterior surface of the polymer article is worn away and the underlying portions or regions are exposed and become the new exterior surface(s), different agents bonded to the molecules of the underlying region are exposed. For example, the top or exterior layer may include polymer molecules including a first agent or combination of agents bonded thereto, and an interior region that is a selected distance below the exterior layer and may include polymer molecules having a second agent or combination of agents, different from the first agent or combination of agents, bonded thereto. Accordingly, as the exterior layer is worn away, the interior region, including the second agent or combination of agents, is exposed.

While the methods, devices and articles disclosed herein are described in relation to medical applications, such methods, devices, and articles are not limited to such applications. The methods, devices and articles may have other uses and may be used in other industries as well.

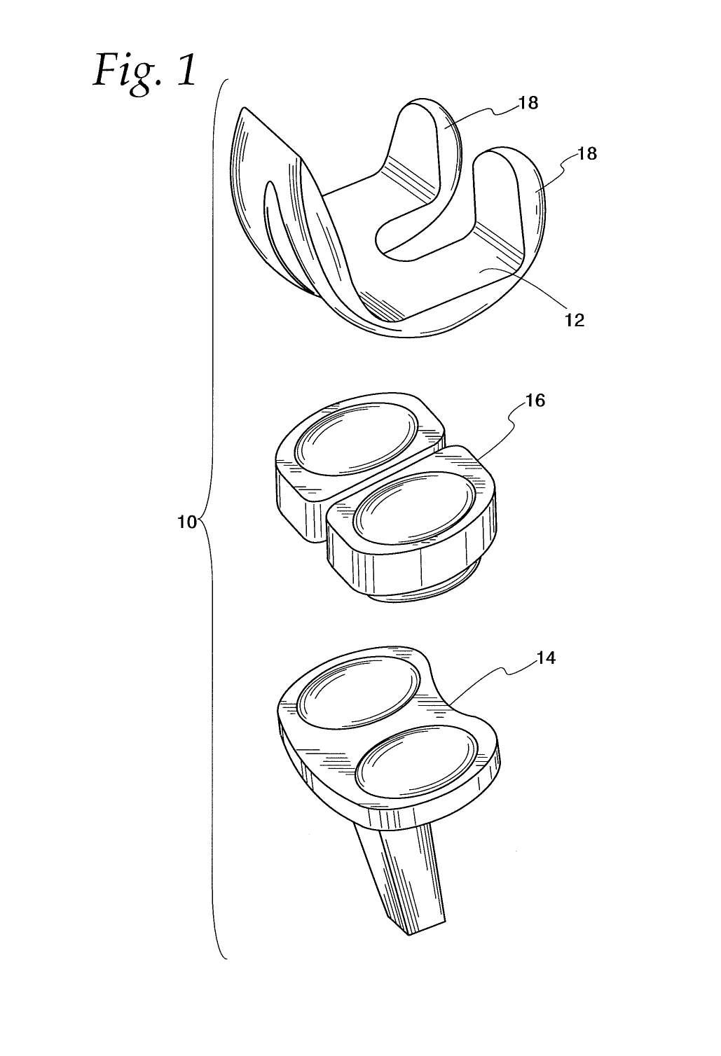

FIG. 1 illustrates one example of a prosthetic implant that may include a polymeric article of the present disclosure. In particular, FIG. 1 shows a prosthetic knee replacement system 10, which includes a femoral implant 12, a tibial implant 14 and polymeric article 16 between the femoral implant 12 and the tibial implant 14. The femoral implant 12 includes a pair of condyle members 18 that bear and articulate against the polymeric article 16. Although polymeric article 16 in this example is shown as a component of a prosthetic knee replacement system, the polymeric articles described herein are not so limited. Polymeric articles of the type described hereinmay be a component of an implant (as shown in FIG. 1), may be the implant itself or may be used in other implant systems, such as, but not limited to, artificial hips and knees, cups or liners for artificial hips and knees, spinal replacement disks, artificial shoulders, elbows, feet, ankles and finger joints, mandibles, and bearings of artificial hearts, etc. The polymeric articles may also be precursors of an article such as the consolidated bulk construct, e.g. slabs, rods or other forms from which the article is made or shaped.

As discussed above, polymeric article 16 includes or is made from a material where the polymer molecules of the material have selected agents bonded thereto. Such polymer molecules may be located at the exterior surface of the article and/or distributed in an interior region of the article. With reference to the exemplary prosthetic implant of FIG. 1, the polymer molecules modified to have agents bonded thereto may be located or present throughout polymeric article 16 so that the polymeric article 16 has agents dispersed substantially across the entire body of the polymeric article. Alternatively, such modified polymer molecules may be located or concentrated in particular sections, portions or layers so that polymeric article 16 only includes agents within selected portions, sections or layers. Further, the polymeric article 16 may have different agents in different layers of the article to form a multilayered construct.

As the condyle members 18 repeatedly and over time articulate against the exterior surface of the polymeric article 16, the exterior surface experiences wear and may eventually be worn away. In accordance with the methods and systems disclosed herein, by having polymer molecules with agents bonded thereto within middle portions, sections or layers of polymeric article 16, as the exterior surface of the polymeric article is worn away, an inner region of polymeric article 16, which includes the modifying agent(s) bonded thereto, becomes the new exterior surface. Thus, the polymeric article may be considered to have a renewable exterior surface. Additionally, different layers or portions of polymeric article 16 can have different agents bonded thereto such that as polymeric article 16 undergoes wear, new layers or sections having different agents (e.g. for providing different properties) bonded thereto will be exposed over time.

The polymeric articles disclosed herein may be made from polymer powders, such as polyethylene, polyaryletherketones, polypropylene, any other suitable polymer, or combinations thereof. One polymer powder that is commonly used in medical implants is UHMWPE. UHMWPE is a semicrystalline, linear homopolymer of ethylene, which may be produced by stereospecific polymerization with a Ziegler-Natta catalyst at low pressure (6-8 bar) and low temperature (66-80 degrees Celsius). The synthesis of nascent UHMWPE results in a fine granular powder. The molecular weight and its distribution can be controlled by process parameters such as temperature, time and pressure. UHMWPE generally has a molecular weight of at least about 2,000,000 g/mol.

Suitable UHMWPE materials for use as raw materials to form the polymeric articles of the present disclosure may be in particulate form such as a powder including flakes or granules or may be provided as a resin. When UHMWPE is used, the polymeric articles may be prepared almost entirely from UHMWPE powder, or may be formed by combining UHMWPE powder with other suitable polymer materials. For example, the UHMWPE may be mixtures of UHMWPEs having different molecular weights. Further, the combinations may be mixtures of UHMWPE with lower molecular weight polyethylene powders, or UHMWPE with other different polymer powders such as, but not limited to, any of the other polymers listed above. In one embodiment the polymeric article may include at least about 50 w/w % UHMWPE.

Examples of suitable UHMWPE powders include GUR 1020 and GUR 1050 available from Ticona, having North American headquarters located in Florence, Ky. Suitable polymer materials for use in combination with the UHMWPE materials may include disentangled polyethylene, high pressure crystallized polyethylene, various other “super tough” polyethylene derivatives or other polymers such as metallocene polyolefins.

The polymeric article may be made from modified polymer powders. Such powders include particles wherein agents are bonded to the polymer molecules that make up the particles and consequently to the particles which are formed from such molecules. The powder of modified particles is further processed to form the article. The polymer powder particles may be flakes, granules or the like. The agents are bonded to the polymer powder particles in a way that leaves functionality of the agent at least substantially intact. In one embodiment, the agents are bonded to the outer surfaces of such particles. In other embodiments, the agents are bonded to the outer surface and interior regions of the particles. The agents may be bonded directly to the polymer molecules of such particles or the agent may be bonded to the molecules of such particles by an intermediary or bridging group, such as a selected reactive group or moiety. The polymer powder having agents bonded thereto may be subjected to one or more of blending with additives, consolidation, crosslinking, annealing, temperature treatments, sterilization processes and additive doping, which may be performed in any combination and in any order.

The agents may be bonded to the polymer powder by any suitable method. In one embodiment, the agent is covalently bonded to the molecules of the polymer powder. In another embodiment, a plasma treatment process may be employed to bond agents to the particles of the polymer powders. Such particles may undergo a plasma treatment in the presence of the agent to bond the agent to the particles. Some methods of plasma polymer modification are disclosed in U.S. patent application Ser. No. 12/938,746 filed on Nov. 3, 2010 which is incorporated by reference herein in its entirety.

The plasma treatment of the polymer powder may take place, for example, in plasma treatment vacuum chamber or in an atmospheric plasma system with a blanketed carrier gas. When a vacuum chamber is utilized, the powder may be place in a rotating drum so that the powder is uniformly exposed to the plasma as the drum is rotated.

In one method of using a vacuum chamber, the polymer powder is placed in the chamber and the chamber is evacuated to a selected pressure, and preferably a relatively low pressure. The pressure may be any suitable pressure depending on the desired application. One or more of selected gases, such as, but not limited to, argon, helium, nitrogen, oxygen, nitric oxide, carbon dioxide, ammonia, amine monomer (primary, secondary or tertiary), acrylic acid or a combination thereof, are then pumped or flowed into the chamber. The gases within the chamber are ionized by, for example, AC, DC or RF voltage, to form a plasma within the chamber. The voltage and/or power level may be such that a plasma is formed from the gases.

Depending on the modifying agent to be bonded to the polymer, the modifying agent may be formed on the polymer with only the selected gases and specific plasma processing parameters. With other modifying agents, one or more of the selected agents, such as a crosslinking group, antioxidant or a biological agent, also may be introduced into the chamber, either before or during the exposure of the polymer powder to the plasma. For example, the polymer powder may be bended with one or more selected agents prior to being exposed to the plasma. In alternative embodiments, one or more selected agents may be added to the plasma treatment system at the same time as the powder is exposed to the plasma or during the exposure the polymer powder to the plasma.

The polymer powder is treated with the plasma in the presence of the one or more selected agents for a selected period of time to bond the agent to the molecules of particles of the powder. The parameters and conditions of the plasma treatment may be selected so that the one or more selected agents bond directly to the molecules of the polymer powder particles. Alternatively, the plasma treatment may result in the bonding of a reactive group or moiety to the polymer molecule, and the agent may be bonded to such reactive group, i.e., the agent may be bonded to the polymer molecule of the polymer powder particle by the reactive group.

In an alternative embodiment, the plasma system may be an atmospheric plasma system. In this embodiment, the powder may be placed into a fluidized bed and then treated with a plasma under atmospheric conditions by any suitable atmospheric methods known in the art. For example, during the atmospheric plasma treatment process, the powder may be passed through a reactive zone containing the reactive gas and plasma. The powder may be cycled through the reactive zone a number of times to produce the desired coverage of reactive groups.

It will be appreciated that the plasma treatment process can be nonspecific in nature. The placement of the agents bonded to the polymer molecules or the surface(s) of the powder particles can be non-specific. The plasma treatment of the polymer powders in the presence of an agent can at least partially be controlled by varying several factors including, but not limited to: (1) the type and shape of the plasma polymer chamber/reactor; (2) the frequency of the discharge excitation voltage; (3) the power of the discharge; (4) the flow rate of gases; (5) the gas pressure within the chamber; (6) the powder temperature; (7) the particle size and geometry; (8) the amount of agent, (9) the type of agent, and (10) the duration of the treatment. These factors may be varied to produce the desired modification for a particular application.

In an alternative embodiment of modifying the polymeric material by the introduction of modifying agents, the particles of the powder may first be treated to bond or graft reactive groups to the particles/particle molecules to provide a reactive surface that is receptive to and may be reacted with selected agents to bond the agents thereto. The reactive groups may include, but are not limited to, one or more of acrylate, amine, amide, imine, imide, hydroxyl, carbonyl, aldehyde, carboxylate, carboxyl, ether, ester, sulfonic, and epoxide groups. The reactive groups may be bonded to the surface of the particles by any suitable process. Preferably, the process used to bond reactive groups to the polymer powder particles bonds the groups to the surface of the particles and leaves the interior of the particle substantially unaltered.

In one example of this alternative embodiment, the polymer powder is plasma treated to bond reactive groups to the surface of the particles of the powder. For example, the polymer powder may be plasma treated in a manner similar to that described above and with a plasma formed from one or more of selected gases, such as, but not limited to, argon, helium, nitrogen, oxygen, nitric oxide, carbon dioxide, ammonia, amine monomer (primary, secondary or tertiary), acrylic acid, or a combination thereof.

Other methods of bonding or grafting reactive groups to the particles may include, but are not limited to, exposing the powder to UV energy or ionizing radiation in the presence of one or more reactive groups or species. When a radiation process is employed and the polymer powders are exposed to high energy radiation or ions, preferably, the process is conducted so as to limit the effects of the radiation or ions to the outer surface of the particles, leaving the interior of the particle substantially undisturbed. In another alternative embodiment, the reactive groups may be bonded to the particles by a grafting reaction in solution. In this process, the solvent, such as toluene, methylene chloride, dimethyacetamide, tetrahydrofurane, carbon tetrachloride, or dimethylsulfoxide, are preferably poor solvents for the polymer, but good solvents for the reactive group.

After the reactive groups have been formed on the surface of the polymer particles of the powder by any suitable process. The particles are exposed to one or more selected agents that react with the reactive groups to bond the agent to the reactive group, thereby bonding the agent to the polymer molecules of the particles. The agents bond with the reactive groups in a way that leaves the functionality of the agent at least substantially intact.

In one embodiment of bonding an agent to a polymer particle of the powder, an antimicrobial agent, such as a metal ion, is bonded to a reactive group of the particle. The metal ions may be, but are not limited to, silver, copper, and zinc. The reactive group may be acid reactive groups such as, but are not limited to, carboxylic acid and sulfonic acid. The particles may be, but are not limited to, UHMWPE, polyether ether ketone (PEEK), polyether ketone ketone (PEKK), polymethyl methacrylate (PMMA) (or other bone cement materials), polyphenylsulfone (PPSU) (for example, Radel© available from Solvay Advanced Polymers, L.L.C. located in Alpharetta, Ga.).

In one embodiment, the polymer powder may be treated with any of the above-described process or any other suitable process to bond an acid reactive group or moiety to the particles of the powder. The polymer powder is then exposed to the antimicrobial agent under conditions that allow the antimicrobial agent to bond to the reactive groups. For example, the acid reactive groups may be neutralized with a metallic base, which includes a metal ion having antimicrobial properties, to bond the metal ion to the relative groups, resulting in a metal salt bonded to the polymer powder particle.

The modified polymer powder is then processed, as described herein, to provide an article having metal salts bonded thereto and selectively dispersed within the article. When the article is implanted into a human or animal body and exposed to bodily fluids, the metal salts will be in solution and form individual ions, which have antimicrobial properties.

For example, as shown in the reaction scheme below, UHMWPE powder is plasma treated with a plasma formed, at least in part, by carbon dioxide (CO₂) gas to bond carboxylic acid groups (CO₂H) (i.e., the reactive groups) to the UHMWPE molecules of the UHMWPE polymer powder particles. The particles are then exposed to silver nitrate (AgNO₃) to react the silver nitrate with the carboxylic acid. This reaction results in silver carboxylate groups (CO₂Ag) bonded to the UHMWPE polymer powder particles and nitric acid (HNO₃).

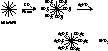

The resulting particles can be processed as described herein to form an implantable article. When implanted, fluids present in the body will cause the silver atoms to disassociate as shown below, resulting in silver ions that can provide an antimicrobial effect in the surrounding environment.

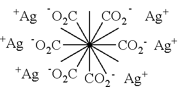

In another embodiment, an agent that promotes bone growth may be bonded to the particles of the polymer powder. For example, the polymer powder may be treated by any suitable process to bond carboxylic acid groups or phosphoric acid groups to the polymer powder particles. For instance, the powder may be plasma treated with carbon dioxide to form carboxylic acid groups (in the manner described above), or treated with phosphorous containing gases to form the phosphoric acid groups. Regardless of the method used, the particles having the acid reactive groups bonded thereto are then neutralized with a calcium compound to form calcium containing salts, which promote bone growth, bonded to the polymer powder particle. The powder then may be processed to form an article having calcium salts bonded thereto and dispersed throughout the article. When the article is implanted into the body, the calcium bonded to the article may provide a bone ongrowth surface.

In yet another embodiment, a polymer can be provided with crosslinking functional groups capable of forming crosslinks in lieu of subjecting the polymer to radiation (e.g., gamma, e-beam). A polymer molecule can have one or more crosslinking functional groups which can react and form chemical bonds with another crosslinking group on a different part of the polymer molecule or an adjacent polymer molecule to form crosslinks. Preferably, a substantial number of all the polymer molecules can have at least one crosslinking group and more preferably a substantial number of the polymers molecules have multiple crosslinking groups to allow formation of multiple crosslinks along the polymer molecule or polymer chain.

In one embodiment, the polymeric article can be constructed with a single polymer type having crosslinking groups i.e., polyethylene, polypropylene, polystyrene, and copolymers such as polypropylene-polyethylene copolymer among others. In another embodiment, the polymeric article can be constructed with more than one polymer types with each polymer having the one or more crosslinking groups such that the crosslinking bonds can be formed between a polyethylene molecule and a polypropylene and even between the same polymer type.

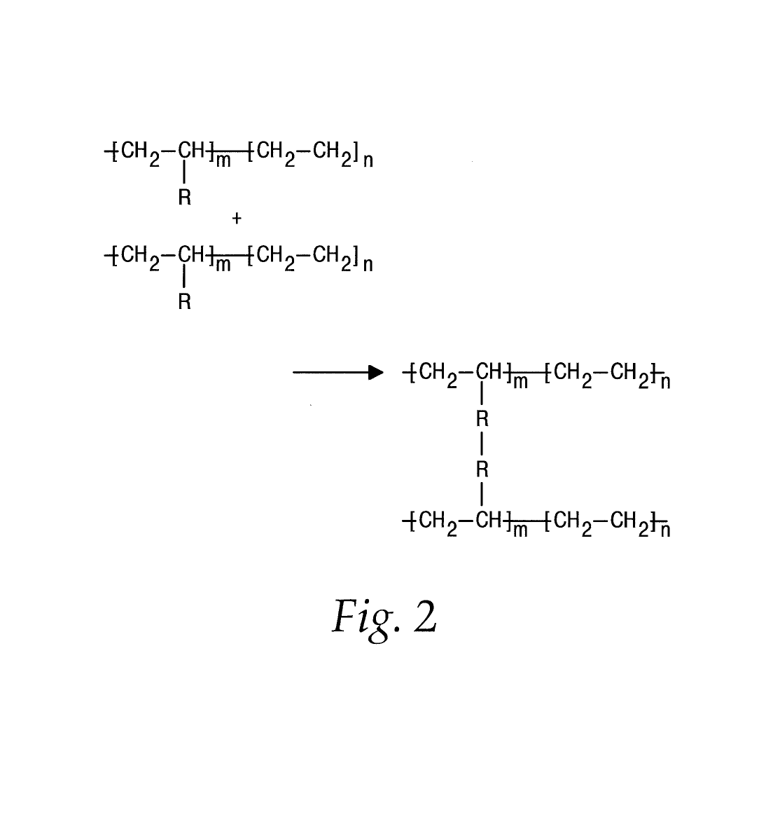

FIG. 2 shows one embodiment where two polyethylene molecules or two different portions of the same polymer molecule each have at least one crosslinking group “R”. The polyethylene can be UHMWPE. The crosslinking groups can react or be made to react to form a chemical bond or crosslink with each other to crosslink the polyethylene polymer molecule(s) without the use of radiation. Avoiding radiation which can cause scissions of the C—H and C—C bond can reduce the formation of persistent free radicals and vinyl groups. Free radicals existing in the crosslinked polymer can reduce the life of the polymer, and vinyl groups are more readily attacked by free radicals or other oxidative attack.

Crosslinking groups can be added to the polymer by methods such as plasma modification. The crosslinking group can also be added during production of the polymer. The degree to which a crosslinking group is added depends on the particular method used. In one embodiment, a majority of the polymer monomers include a crosslinking group. In another embodiment, a minority of polymer monomers include a crosslinking group. In one embodiment “m” in the polyethylene polymer of FIG. 2 can be from about 0.1 to about 10% in terms of mole percent.

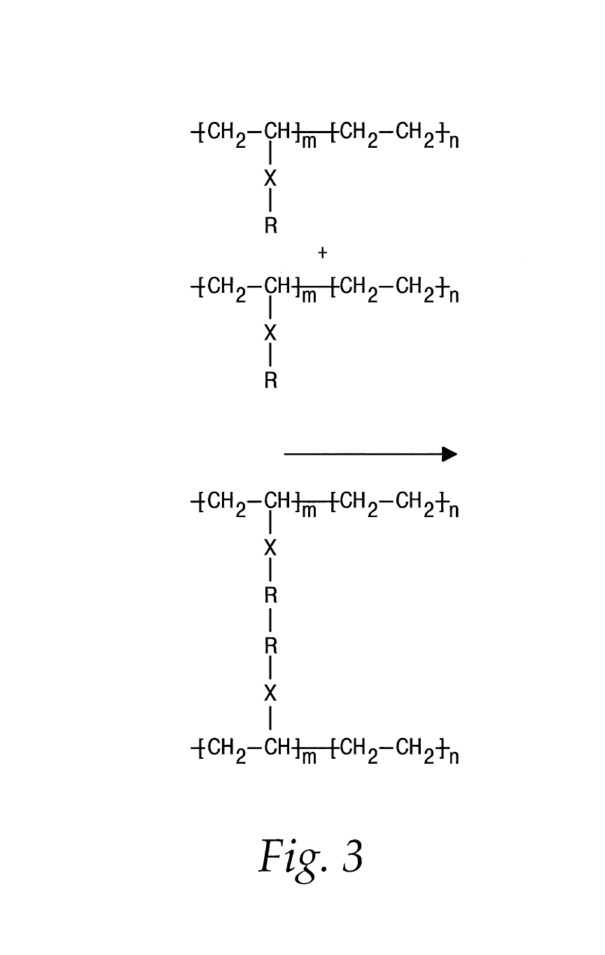

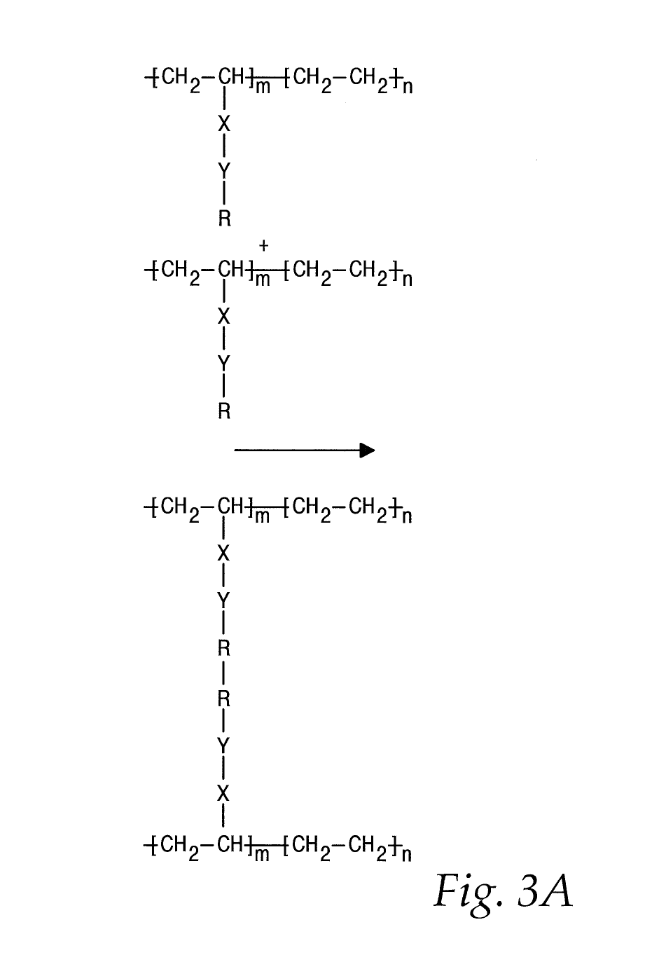

In another embodiment as shown by way of example in FIG. 3, the polymer can include a bridging group “X” between the crosslinking group “R” and the polymer backbone. Bridging group “X” can be any element or compound which readily forms a bond with the carbon backbone of the polymer and the crosslinking group. The bridging group can be selected based on the desired degree of spacing, bonding preferences or other parameters. The bridging group can be selected from but not limited to acrylate, amine, amide, imine, imide, hydroxyl, carbonyl, aldehyde, carboxylate, carboxyl, ether, ester, sulfonic, epoxide, alkanes, alkyl ethers, alkyl esters, perfluoroalkyl, and aromatic groups, also oligomers or polymers such as low molecular weight polyethylene glycol, polyethylene, polymethacrylic acid, polyacrylamide. In yet another embodiment shown in FIG. 3A, the polymer can include a second bridging group “Y” which can assist in bonding crosslinking group “R” to bridging group “X” or to just provide additional spacing. In one embodiment the bridging group “Y” can be an oxygen.

Similarly in another embodiment, a crosslinking group is provided on one or more propylene monomers of a polymer. Preferably, the crosslinking group replaces a tertiary hydrogen of at least some of the polypropylene monomers. Replacing a tertiary hydrogen of the propylene reduces the reactivity of the polymer and makes it less susceptible to free radical and/or other oxidative attack. Since raw polymer such a polypropylene can be stored for long periods before crosslinking the polymer, free radical and oxidative breakdown may build such that when and if the polymer is crosslinked and formed into an article its life may have been shortened by the oxidative damage that may have already occurred. In addition, free radical accumulation can shorten the incubation period which can result to accelerated oxidation.

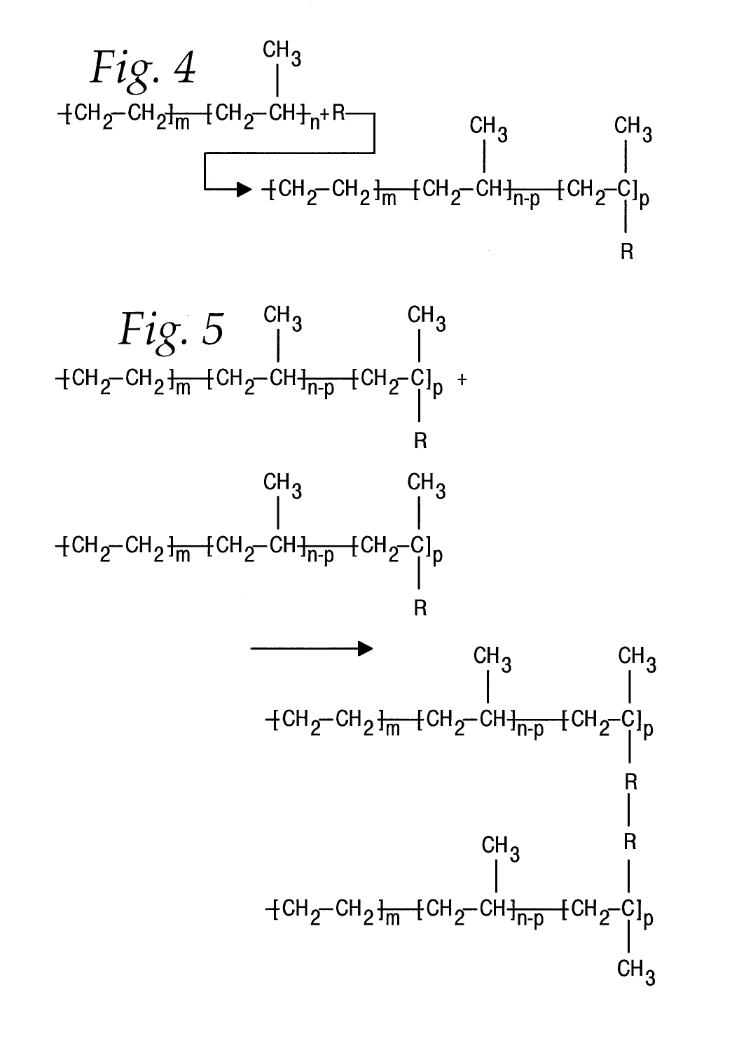

One embodiment of a crosslinked polyethylene polypropylene (PE/PP) copolymer using polymers having crosslinking groups is shown in FIG. 5. Crosslinking of a PE/PP copolymer using crosslinking groups instead of radiation crosslinking can have several benefits. As shown in FIG. 4, crosslinking group “R” can be bonded to the branched carbon and replace the tertiary hydrogen of a particular percentage of the polypropylene monomer. This elimination of a tertiary hydrogen can reduce the reactivity and vulnerability to oxidative and/or free radical attack of the PE/PP copolymer which can prolong the life of the copolymer and any article made therefrom. In one embodiment, crosslinking group “R” can be added from about 0.1 to about 10% of the polypropylene monomers. In another embodiment, crosslinking group “R” can be added such that the polypropylene monomers having a crosslinking group make up from about 0.1 to about 10% mole percent of the polymer.

As shown in FIG. 5, when one copolymer having a crosslinking group “R” is reacted with another PE/PP copolymer chain having the same crosslinking group the reaction produces a crosslinked PE/PP copolymer.

Crosslinking provided via the bonding of crosslinking groups “R” may be preferable over radiation treatment by preventing or reducing the formation of vinyl groups in some of the ethylene monomers, and tertiary hydrogen if the crosslink were to form between the ethylene monomer of the PE/PP copolymer chain produced through radiation treatment. Accordingly, the crosslinked PE/PP copolymer formed by bonding of crosslinking groups can result in a reduced number of tertiary hydrogens prior to crosslinking which can extend the life of the copolymer during long term storage and the reduction or elimination of vinyl groups and tertiary hydrogen formation following crosslinking which can also extend the life of the polymer and/or elimination of polymeric free radicals typically created by irradiative crosslinking.

In the embodiment shown in FIG. 4, “m” can be from about 1% to about 10% by mole percent, “n” can be from about 90% to about 99% by mole percent and “p” can be from about 1% to about 80% depending on a number of factors such as the method used to modify or produce the polyethylene polypropylene copolymer.

In one embodiment, the heating can be performed in a heating step. Alternatively, the heating may be provided as a result of any consolidation steps performed on the polymer or polymer resin such as compression molding, sintering, injection molding or extrusion. In another embodiment the heating can be applied in addition to any consolidation steps.

In another embodiment, the crosslinking functional groups can be selected from but not limited to sulfonic acid groups, trifluorovinyl, phosphoric acid groups, carboxylic acid groups, epoxides, and cyano groups. Each of these crosslinking groups can react and form bonds with each other in (or under the influence of) a thermal reaction process without additional reactants. The heat required to initiate the reaction and form crosslinking bonds can be at or below the melting point of the polymer. In one embodiment, the heat required to react the crosslinking groups to form crosslink bonds can be at or below 180° C., preferably at or below 150° C., and more preferably at or below 120° C.

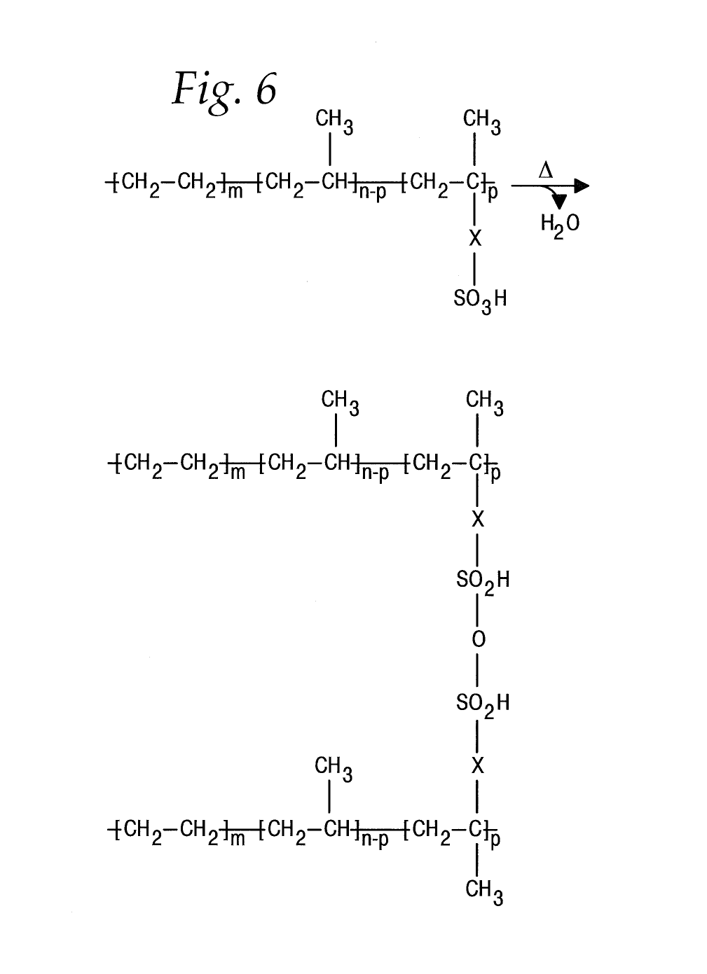

In yet another embodiment, a thermally crosslinked PE/PP copolymer is provided. As shown in FIG. 6, a PE/PP copolymer can have a sulfonic acid crosslinking group bonded to the polypropylene monomer of the copolymer chain via a bridging group “X”. Alternatively, the sulfonic acid crosslinking group can be bonded directly to the copolymer either on the PE or PP monomer. Upon application of heat to raise the temperature to between about 110 to about 250° C. for between about 1 to 5 hours (requisite heat energy), the crosslinking groups undergo a dehydration reaction losing water and forming the crosslinking bond. Preferably the temperature of the polymer can be raised from about 110° C. to about 180° C. to initiate the crosslinking reaction, and more preferably from about 110° C. to about 150° C. and even more preferably from about 110° C. to about 130° C. In one embodiment bridging group can be a methyl group. To provide additional spacing, the bridging group “X” can have a benzene ring or an additional bridging group can be added.

In one embodiment “m” can be from about 1 to about 10 mole percent and the sulfonic acid group can be present on at least from 0.1 to about 2% of the PP monomer. In the embodiment shown in FIG. 6 “m” can be from about 1% to about 10% by mole percent, “n” can be from about 90% to about 99% by mole percent and “p” can be from about 1% to about 80% depending on a number of factors such as the method used to modify or produce the polyethylene polypropylene copolymer.

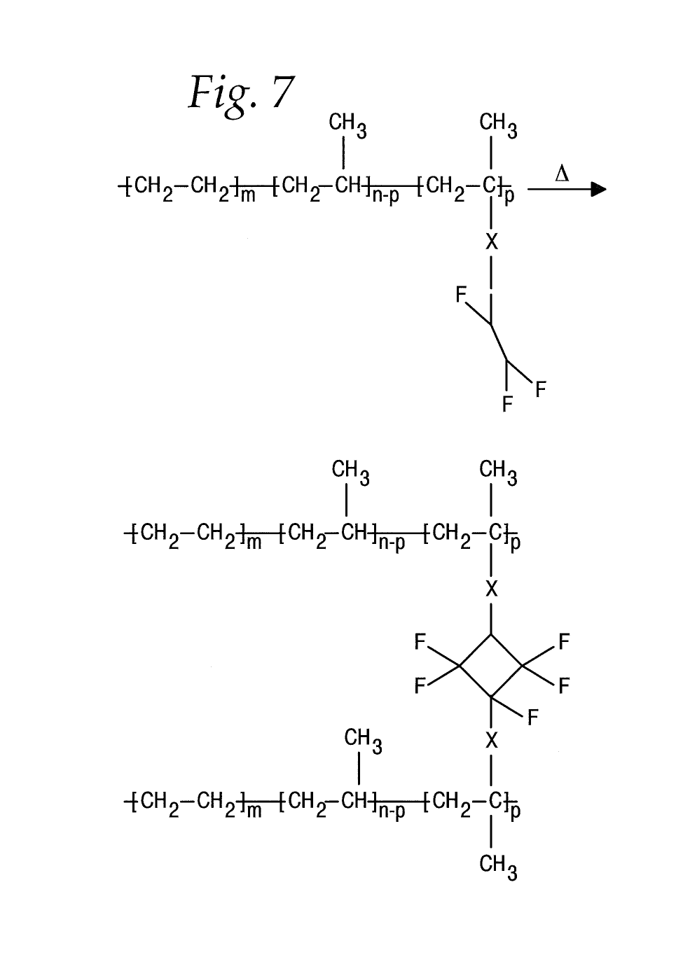

FIG. 7 shows another embodiment of a thermally crosslinked PE/PP copolymer. As shown in FIG. 6, a PE/PP copolymer can have a trifluorovinyl crosslinking group and can be bonded to the polypropylene monomer of the copolymer chain via a spacer group “X”. Alternatively, the trifluorvinyl crosslinking group can be bonded directly to the copolymer. Upon application of heat to raise the temperature to between about 120 to about 150° C. for about 1 to 5 hours, the crosslinking groups can undergo a cyclodimerization reaction to form the crosslinking bond. Preferably the temperature of the polymer can be raised to about 110° C. to about 180° C. to initiate the crosslinking reaction, more preferably from about 110° C. to about 150° C. and even more preferably from about 110° C. to about 130° C.

In one embodiment, bridging group can include a benzene ring. In one embodiment, the trifluorovinyl crosslinking group can be present on from about 0.1% to about 2% of the PP monomer. In another embodiment “m” can be from about 1% to about 10% by mole percent, “n” can be from about 90% to about 99% by mole percent and “p” can be from about 1% to about 80% depending on a number of factors such as the method used to modify or produce the polyethylene polypropylene copolymer

Where the crosslinking group is a cyano crosslinking group the temperature can be from room temperature of about 20° C. to about 50° C. to initiate the crosslinking reaction, preferably from about 25° C. to about 40° C. and more preferably from about 25° C. to about 30° C.

Processing of Modified Polymer Powder

After the agents have been bonded to the particles of the polymer powder by any suitable process, the modified polymer powder may be processed further to form an implantable polymeric article. As discussed above, the polymer powder having agents bonded to the particles of the powder thereto may be subject to one or more of blending with additives, consolidation, crosslinking, annealing, temperature treatments, sterilization processes and additive doping, which may be performed in any combination and in any order.

Optionally, the modified polymer powder including agents may be further blended with additional agents, such as, but not limited to, antioxidants, antibiotics, antimicrobials or anti-inflammatories. The antioxidant may be, for example, vitamin E. Such additional agents, in some instances, may be leachable out of the final formed polymeric article, if desired. Accordingly, the polymeric article may include non-leachable agents (the agents bonded to the molecules of the polymeric article and are not readily drawn out of the article) and leachable agents (agents that are not bonded to the polymeric article and are capable of leaching out of the article).

The blended or unblended modified polymer powder may then be consolidated and/or compressed into a suitable form for use as (or as part of) a prosthetic device or other implant. Suitable compression and/or consolidation techniques include, for example, compression molding, direct compression molding, hot isostatic pressing, ram extrusion, high pressure crystallization, injection molding, sintering or other conventional methods of compressing and/or consolidating polymer powders. This compression or consolidation techniques may produce enough heat to intiate crosslinking groups to react and form crosslinks, or additional heating steps may performed. If desired, the polymeric article formed from the compressed/consolidated polymeric article may be further processed or manufactured by crosslinking, annealing, melting, heating, cooling, doping with antioxidant, doping with biological agents, milling, machining, drilling, cutting, assembling with other components, and/or other manufacturing or pre-manufacturing steps conventionally employed to manufacture implants from polymer. For example, the modified powder may be subject to any of the processes of forming an article disclosed in U.S. Patent Application Publication No. US2010/0029858, published Feb. 4, 2010, and US2009/0118390, published May 7, 2009, which are incorporated herein by reference.

A multilayered construct or article may be made during the compression molding process. For instance, polymer powders modified to have selected properties or characteristics may be selectively located in particular regions within the mold. For example, to make a layered construct, a first polymer powder having a first type or types of agents may be located at the bottom of the mold to form a first layer. A second polymer powder having different agents or no agents bonded thereto (raw unmodified polymer powder) may be placed on top of the first layer to create a second layer. In other embodiments, several layers of modified and/or unmodified polymer powders may be located in the mold. Further, placement of the polymer powders is not limited to layers. The polymer powders, having different agents now incorporated therein, may be selectively placed in different regions or portions of the mold. Once the polymer powders have been placed in the mold, the powder is compression molded to form an article for use in or as a medical implant or a bulk material that can be shaped into such an article.

When polymer powders, such as UHMWPE, are consolidated, the polymer molecules located at the grain boundaries of the polymer powder particles (i.e., at the surface of the particle) entangle with polymer molecules at the grain boundary of adjacent particles. Although the polymer chains migrate and intermingle, the grain boundaries are substantially retained. The grain boundaries of a consolidated polymeric UHMWPE article represent the areas most susceptible to oxidation. When an antioxidant is bonded to the polymer particles as described herein, preferably, the antioxidant is bonded to the surfaces of the polymer particles so that after consolidation, the antioxidant will be located at the grain boundaries to create greater oxidation resistance in such areas.

Prior to and/or after processing the implant as discussed above, the polymer may be crosslinked by any suitable crosslinking process. For example, the polymer may be crosslinked by exposure to radiation at a high radiation dose and/or a dose rate sufficient to form a crosslinked polymer in addition to any crosslinking produced through the modified polymers having crosslinking groups or in lieu of otherwise modified polymers not including crosslinking groups. The radiation may be, for example, gamma or electron beam irradiation. In one embodiment, the polymeric article may be exposed to electron beam irradiation at a dose rate of between about 25 kGy/min and about 240 kGy/min for a total dose of between about 50 kGy and about 200 kGy. In certain embodiments, the desired radiation dose may be achieved in a single exposure step at a high dose rate. In other embodiments, a series of high dose rate irradiation steps may be employed to expose the polymer to a desired dose of radiation. The crosslinking may be conducted at any time from powder to implant. The crosslinking also may occur before or after powder modification as disclosed herein and may be used in conjunction with other manufacturing processes applied to the polymeric article. Further, prior to irradiation, the polymer may be preheated.

In certain embodiments, the radiation source is electron beam radiation. Electron beam radiation exposure may be performed using conventionally available electron beam accelerators. One commercial source for such an accelerator is IBA Technologies Group, Belgium. Suitable accelerators may produce an electron beam energy between about 2 and about 50 MeV, more particularly about 10 MeV, and are generally capable of accomplishing one or more of the radiation doses and/or dosage rates reported herein. Electron beam exposure may be carried out in a generally inert atmosphere, including for example, an argon, nitrogen, vacuum, or oxygen scavenger atmosphere. Exposure may also be carried out in air under ambient conditions according to one embodiment. Gamma and x-ray radiation may also be suitable for use in alternate embodiments. The processes described herein are not necessarily limited to a specific type of source of radiation.

In another embodiment, the functional or reactive group incorporated into the polymer powder may not only serve as binding location for agents or for crosslinking, but may also serve other functions, properties or characteristics, such as increased lubricity, hydrophobicity, hydrophilicity, or wettability.

The polymeric article formed from consolidation of the modified powder may also be subject to annealing. When annealing is employed, the polymeric article may be annealed at a temperature of between about 100° C. and about 160° C. for a time period of between about 2 hours and about 40 hours. This may produce sufficient heat to intiate reactions of the functional groups such as crosslinking. The annealing may be used in conjunction with other manufacturing processes applied to the polymeric article. Alternatively or additionally, the crosslinked polymer may be subjected to the mechanical annealing processes reported in U.S. Pat. No. 6,852,772 to Muratoglu, which is incorporated herein by reference. In one embodiment, however, no pre- or post-irradiation temperature and/or annealing treatments are performed. In another embodiment, the polymeric article may be subject to an irradiation process and then annealed.

As part of the implant manufacturing process, additional components may be combined with the polymer at any time during the process reported herein. In one embodiment, tribological components such as metal and/or ceramic articulating components and/or preassembled bipolar components may be combined with the polymer. In other embodiments, metal backing (e.g. plates or shields) may be added. In further embodiments, surface components such a trabecular metal, fiber metal, beads, Sulmesh® coating, meshes, cancellous titanium, and/or metal or polymer coatings may be added to or joined with the polymer. Still further, radiomarkers or radiopacifiers such as tantalum, steel and/or titanium balls, wires, bolts or pegs may be added. Further yet, locking features such as rings, bolts, pegs, snaps and/or cements/adhesives may be added. These additional components may be used to form sandwich implant designs, radiomarked implants, metal-backed implants to prevent direct bone contact, functional growth surfaces, and/or implants with locking features. The finished implant is typically in either gas permeable packaging or barrier packaging utilizing a reduced oxygen atmosphere.

A variety of implants, and in particular endoprosthetic joint replacements, may be prepared by employing the methods reported herein. Examples of such implants include artificial hips and knees, cups or liners for artificial hips and knees, spinal replacement disks, artificial shoulder, elbow, feet, ankle and finger joints, mandibles, and bearings of artificial hearts.

### EXAMPLES

The following non-limiting examples illustrate various features and characteristics of the present invention, which is not to be construed or limited thereto.

### Example 1

In the various Samples below, UHMWPE powder resin GUR 1050 brand powder available from Ticona, having North American headquarters located in Florence, Ky. were used.

Sample A

GUR 1050 powder was plasma treated with CO₂ by PVA TePla America, Corona, Calif. to form a UHMWPE powder that included carboxylic acid reactive groups bonded to the particles of the powder. 100 g of the treated powder was placed into a beaker and 300 ml of 2% ethanolic silver nitrate solution was added to the beaker. The mixture was heated to 50° C. for 3 hours. The powder was filtered from the solution. The powder was rinsed with ethanol, rinsed with deionized water, and then rinsed again with ethanol. The powder was dried in a vacuum oven ensuring that the powder was not exposed to light. Table 1 generally lists the processing parameters of Sample A. The powder was then consolidated by compression molding to produce a puck or generally cylindrical polymeric article having a diameter of 2.5 inches and a height of 2 inches.

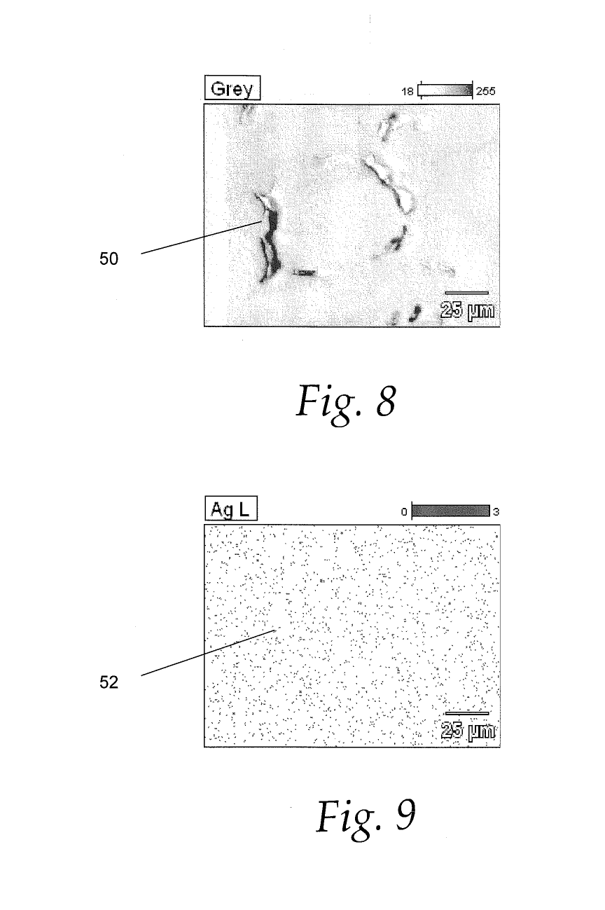

FIGS. 8 and 9 are SEM images of a portion of an article made by the above process. FIG. 8 is an image showing a grain boundary 50. FIG. 9 is an image of the same portion shown in FIG. 8 wherein the image has been adjusted to show the location of silver atoms 52. As shown in FIG. 9, the silver atoms are dispersed throughout the article made by the above process.

Samples B-H

Samples B-H were prepared as comparative samples, and Table 1 sets forth the processing parameters for such Samples. Sample B was virgin GUR 1050 powder that did not undergo any treatments.

For Samples C-H, the GUR 1050 powder was plasma treated by the gases listed in Table 1. The plasma treatment process was carried out by PVA TePla America, Corona, Calif. In all of these samples, the plasma treated powder was then consolidated by compression molding to produce a puck or generally cylindrical polymeric article having a diameter of 2.5 inches and a height of 1.5 inches. In sample H, during the compression molding process, the powder was held under compression and at an elevated temperature for an extended period of time.

TABLE 1

| | RAW | PLASMA | |
|---|---|---|---|
| | MATERIAL | TREATMENT | ADDITIONAL |
| SAMPLE | GUR | GASES | TREATMENT |
| A | GUR 1050 | CO₂ | React with AgNO₃ |
| B | GUR 1050 | N/A | N/A |
| C | GUR 1050 | Acrylic Acid | N/A |
| D | GUR 1050 | Allylamine | N/A |
| E | GUR 1050 | N₂O | N/A |
| F | GUR 1050 | CO₂ | N/A |
| G | GUR 1050 | NH₃ | N/A |
| H | GUR 1050 | NH₃ | Extended period of |
| | | | compression/temperature |
| | | | elevation during |
| | | | compression molding |

### Results

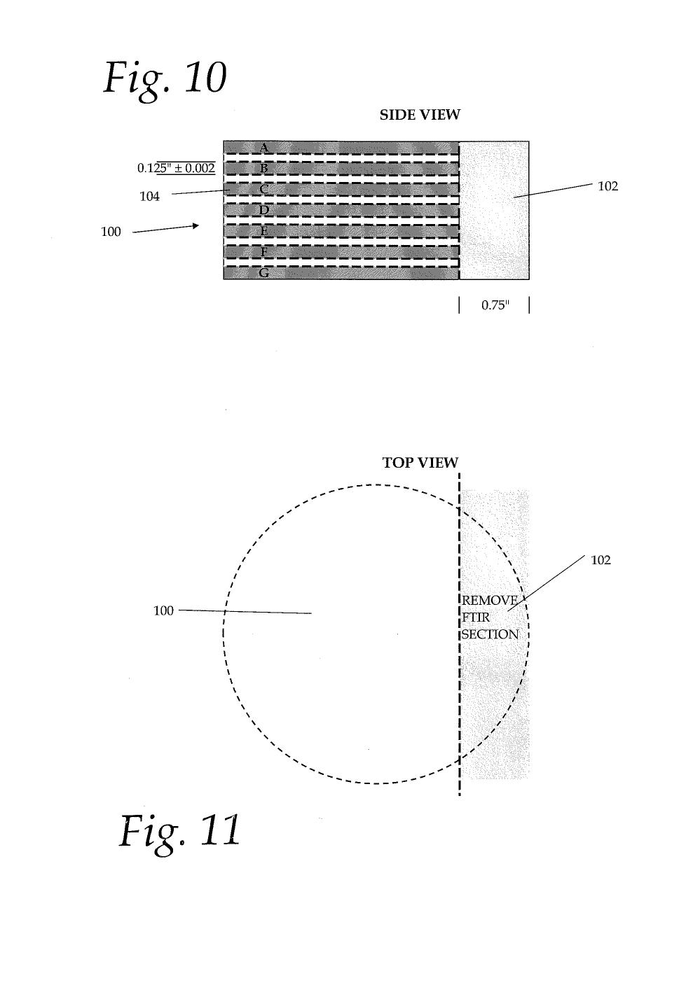

Tests were preformed on the above samples to determine the physical properties of the consolidated and processed polymer materials of the above Samples. In particular, tests were conducted on sections of material taken from middle sections of the above-described compression molded puck. FIGS. 10 and 11 illustrate one example of a puck 100. A 0.75 inch portion 102 of the puck as measured from an edge of the puck was removed. Referring to FIG. 10, the puck 100 was then machined to create a plurality of flats 104 from the middle section of the puck. The flats 104 had a thickness of about 0.125±0.002 inches.

Tensile Test Results

The tensile properties of the samples A-H described above were tested according to ASTMD638-02a. Tensile bars test specimens were punched from flats 104.

Tensile properties of each Sample were determined from the average of 10 runs. An Instron Model 3345 Test System available from Instron, Norwood, Mass., USA was used to test the tensile properties of each sample. The results are listed in Table 2.

TABLE 2

| | ULTIMATE | % STRAIN AT | ZERO SLOPE |
|---|---|---|---|
| | TENSILE | AUTOMATIC | YIELD STRESS |
| SAMPLE | STRENGTH (MPA) | BREAK (%) | (MPA) |
| A | 31 | 213 | 22 |
| B | 54 | 425 | 22 |
| C | 31 | 213 | 22 |
| D | 33 | 231 | 22 |
| E | 47 | 325 | 25 |
| F | 43 | 301 | 25 |
| G | 33 | 179 | 33 |
| H | 40 | 226 | 40 |

Contact Angle Test Results

Contact angle measurements, which measure the angle between the surface of a liquid solvent (e.g. water, serum) and the surface of the polymer substrate at the line of contact, were conducted on samples A-H in order to test the lubricity of the exterior surface layer of the puck. In general, the lower the contact angle, the more wettable the surface, which indicates greater lubricity with the solvent. In the present investigation, deionized water was used as the solvent, and the contact angles were measured using a Kruss DA 100. The contact angle test results are shown in Table 3.

TABLE 3

| SAMPLE | CONTACT ANGLE |
|---|---|
| A | 90 |
| B | 90 |
| C | 76 |
| D | 78 |
| E | 58 |
| F | 65 |
| G | 58 |
| H | 52-80 |

Further, for Samples A, D and C, tensile bars were cut from the puck and contact angle test were preformed on the tensile bars. In the present investigation, deionized water was used as the solvent, and the contact angles were measured using a Kruss DSA 100. The contact angle test results are shown in Table 4.

TABLE 4

| SAMPLE | CONTACT ANGLE |
|---|---|
| A | 80 |
| D | 70 |
| C | 65 |

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. An implantable medical device comprising:
a polymeric article having an outer surface and an interior region, the article comprising polymer molecules with at least one modifying agent bonded thereto, and wherein the polymer molecules are distributed within at least a portion of the interior region.

2. The implantable medical device of claim 1 wherein the at least one modifying agent is bonded directly to the polymer molecules.

3. (canceled)

4. The implantable medical device of claim 1 wherein the polymer molecules include a reactive group and the at least one modifying agent is bonded to the reactive group.

5. (canceled)

6. The implantable medical device of claim 1 wherein the polymer molecules comprise at least one of ultra high molecular weight polyethylene, polypropylene or polyethylene polypropylene copolymer.

7. The implantable medical device of claim 1 wherein the polymer article comprises from 0.1 to 10 mole percent of polymer molecules having the at least one modifying agent bonded thereto.

8. The implantable medical device of claim 1 wherein the at least one modifying agent is an antioxidant.

9. The implantable medical device of claim 8 wherein the antioxidant is tocopherol.

10. The implantable medical device of claim 1 wherein the at least one modifying agent is a biological modifying agent.

11. The implantable medical device of claim 10 wherein the biological modifying agent is selected from the group consisting of antimicrobials, antibiotics, anti-inflammatories, and combinations thereof.

12. (canceled)

13. The implantable medical device of claim 1 wherein the at least one modifying agent is a thermally activated crosslinking group for chemically bonding to another thermally activated crosslinking group to crosslink the polymer molecules.

14. The implantable medical device of claim 13 wherein the crosslinking group is selected from the group consisting of a sulfonic acid group, a fluorovinyl group, a phosphoric acid group, a carboxylic acid group, an epoxide group, a cyano group, and combinations thereof.

15-19. (canceled)

20. The implantable medical device of claim 13 wherein at least one monomer of at least one of the polymer molecules has the following formula: 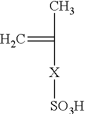
wherein X is the reactive group.

21. The implantable medical device of claim 13 wherein the crosslinking group is a fluorovinyl group and at least one monomer of at least one of the polymer molecules has the following formula: 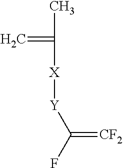
and wherein the reactive group X is selected from the groups consisting of alkanes, alkyl ethers, alkyl esters, perfluoroalkyl, aromatic, oligomers, polyethylene glycol, polyethylene, polymethacrylic acid, and polyacrylamide, and the second reactive group Y is either nothing or an oxygen.

22-24. (canceled)

25. The implantable medical device of claim 1 wherein the at least one modifying agent is a non-leachable modifying agent.

26. (canceled)

27. A polymer material for manufacturing a medical implant, comprising:
a polymer powder including particles; and
a modifying agent bonded to the particles.

28. The polymer material of claim 27 wherein the modifying agents comprises crosslinking groups which are bonded to each other by a thermally initiated reaction of the crosslinking groups.

29. The polymer material of claim 28 wherein the polymer powder includes monomers having a branched carbon and the crosslinking groups are bonded to the branched carbon.

30. The polymer material of claim 28 wherein the polymer material is substantially free of vinyl groups and tertiary hydrogens.

31. The polymer material of claim 28 wherein the reaction between the crosslinking groups is selected from the group consisting of a cyclodimerization reaction, a dehydration reaction, a condensation reaction, or an addition reaction.

32-34. (canceled)

35. A method of manufacturing an implantable medical device formed of a polymeric article, comprising the steps of:
bonding one or more modifying agents to molecules of polymer particles; and
consolidating the polymer particles to form a polymeric article.

36. The method of claim 35 further comprising the steps of:
reacting the modifying agents wherein the one or more modifying agents are crosslinking groups which crosslink polymer molecules.

37. The method of claim 36 wherein reacting the crosslinking groups includes a reaction selected from the group consisting of a cyclodimerization reaction, a dehydration reaction, a condensation reaction, or an addition reaction.

38. The method of claim 35 wherein the step of bonding one or more of the modifying agents to molecules of the polymer particles comprises:
exposing the polymer particles to at least one of a plasma, UV energy, and ionizing radiation, wherein the polymer particles are exposed in the presence of the one or more modifying agents to bond the one or more modifying agents to the polymer particles.

39. (canceled)

40. The method of claim 35 wherein the step of bonding one or more of the modifying agents comprises the steps:
bonding a reactive group to particles of a polymer; and
bonding a modifying agent to the reactive group.
